(19) United States
(12) Patent Application Publication
(10) Pub. No.: US 2019/0083766 A1
(43) Pub. Date: Mar. 21, 2019

(54) Ureteral stent with a non-irritating and shock-absorbing bladder anchor

(71) Applicant: Rupesh Desai, San Jose, CA (US)

(72) Inventor: Rupesh Desai, San Jose, CA (US)

(21) Appl. No.: 16/137,304

(22) Filed: Sep. 20, 2018

Related U.S. Application Data
(60) Provisional application No. 62/561,577, filed on Sep. 21, 2017.

(51) Int. Cl. A61M 27/00

(52) U.S. Cl. CPC A61M 27/008; A61F 2002/048

(57) ABSTRACT

A stent having an elongated tubular configuration consisting of at least one anchor section to inhibit migration. Said anchor section is fabricated by an expandable mesh of woven metallic or polymeric elements. The anchor is configured to minimize contact abrasion and irritation to the entrance of the lumen being stented, as well as to enervated tissue surrounding that entrance, as well as to better absorb forces and increase retention as the lumen is stretched.

### FIELD OF INVENTION

The present invention relates to a novel non-irritating and shock-absorbing ureteral stent anchor. More particularly, the invention relates to the design of the anchor to enable it to minimize or eliminate irritation and discomfort associated with the bladder anchor of a ureteral stent.

### BACKGROUND

The double-J or pigtail ureteral stent is widely used to maintain ureteral patency post-endoscopic procedures, as well as for relief of longer-term obstructive disease. The pigtail catheter provides a self-retaining capability due to a coil design at proximal and distal ends that work to securely anchor the stent in the urinary tract between the renal pelvis and the bladder. These anchors prevent stent migration proximally or distally despite urinary flow, patient movement, and ureteral peristalsis.

The stent anchor may irritate the bladder, causing patient discomfort and a need to pass urine frequently. Referred pain as well as pain upon urination is common, as is hematuria (blood in urine) in severe cases. It is believed that the relatively small bearing surface of the existing stent shaft contributes to the irritation.

These stent-related symptoms may impact large populations of patients. They include voiding symptoms including frequency, urgency, dysuria, incomplete emptying; flank and suprapubic pain; incontinence, and hematuria. To lessen these symptoms, a wide array of anchor designs and alternative stent tube materials has been proposed. Anchor designs other than the pigtail include the “Tailstent” with long floppy strands extending into the bladder (U.S. Pat. No. 6,656,146), stents utilizing a collapsible Mallecot type structure (U.S. Pat. No. 7,470,247), and stents with an expandable coil anchor (U.S. Pat. No. 4,531,933) or an expandable coil perpendicular to the stent axis (U.S. Pat. No. 6,620,202). Of significant interest are ureteral and urethral stents utilizing spherical, ellipsoidal and convex braided mesh anchors in the bladder and also in the GI tract (U.S. Pat. Nos. 6,395,021 and 6,558,350).

Several ureteral and other proposed stents are made entirely of a self-expanding mesh for the simultaneous purposes of self-anchoring and dilation of a duct or vessel. These are principally utilized in a blood vessel, and in the GI tract. The blood vessel devices are anchored in position by the wires of the mesh pressing into the wall of the vessel, causing extrusion of tissue into the openings of the mesh. This approach typically works much better in a blood vessel rather than in a GI lumen or a ureter where there is significant peristalsis. Some of the GI devices are anchored in this manner, more commonly others, used with one or more ends protruding into an expanded space, are anchored in a manner similar to a ureteral stent. Those devices depend on an enlarged section to prevent the stent from being pushed or pulled out of the lumen being treated. The typical expanded shape is a sphere or ellipse or an abrupt increase in diameter, one that is a natural result of a self-expanding device in an unconstrained space.

Use of a self-expanding mesh stent with spherical, elliptical, or abrupt or tapered convex anchor shapes, although simple to implement, create several difficulties. Most important, the bearing surface against the delicate tissue surrounding the ureteral entrance consists of small wires. Self-expanding braid, optimized for self-expansion and dilation of a constricted lumen are likely too stiff to be tissue friendly. When small diameter wires are pulled against the tissue by a change in patient position or by physiologic movement such as peristalsis, these small wires can cause local damage/abrasion via the “cheese-cutter” effect.

Secondly, the tissue in and immediately adjacent to the ureteral orifice in particular is known to be heavily enervated and sensitive to trauma. A spherical or elliptical mesh anchor, even if soft enough to provide some shock absorption, will not only irritate the tissue immediately adjacent to the ureteral entrance, but may be drawn into the duct itself, scraping the proximal intima with its wire mesh. If too soft, the anchor may not provide sufficient anchoring force. The difficulty in trading off avoidance of tissue damage, good shock absorption, and sufficient retention is the reason that these types of stents are not currently employed in the bladder and ureter, although they have some usage in the GI tract. The current invention addresses all three of these challenges in such a manner as to provide improvement in all three areas over current “double-J” products as well as prior self-expanding mesh anchors.

### BRIEF SUMMARY OF INVENTION

The present invention of a novel ureteral stent device is comprised of a polymer tube (of size 3 to 4 French for pediatric and 4 French and higher for adult) with a traditional pigtail shape tapered anchor for anchoring in the kidney, and a unique soft bladder anchor made out of a fine metal or polymeric braid. The braid is preferably made out of metallic alloy such as Nitinol (NiTI) material. NiTi can be a soft shape-memory alloy which may be shape-set into a desired form, such as an ovoid or sphere or cup with relatively high surface area as compared to stent tubing. NiTi is well known for its ability to return to its pre-set shape.

The braid alloy, wire size, and pick count are precisely selected in order to create a soft and non-irritating anchor in the bladder. The braid is heated to shape-set into a secondary shape such as a single or double spherical shape, oval, donut, cone, cup, etc. Sufficient flexibility is designed into the braided structure to allow it to deform to act as a retaining shock absorber, while still being a relatively soft, non-irritating bladder anchor.

The stent anchor of the present invention is formed of a cup or concave mesh anchor structure which forms a contact region spaced radially outwardly from the shaft or body of the ureteral stent. Thus, its annular ring bears on the tissue close to but not immediately adjacent to the bladder entrance of the ureter (commonly called the ureteral orifice or UO). This structure takes full advantage of the theoretical benefits of the prior spherical or elliptical mesh anchors which include relatively large diameter expansion, unrestricted flow of urine through the mesh, and large bearing surface against the bladder tissue as compared to the prevalent “Double J” design. However, the annular contact ring of the concave/cup configuration avoids contact of the anchor with tissue immediately adjacent to the ureteral orifice, with the orifice itself, as well as with the proximal ureter. To avoid trauma to the highly enervated bladder tissue adjacent to the ureteral orifice the diameter of non tissue contact should be a minimum of three times the diameter of the stent shaft, that shaft diameter typically defining the diameter of the ureteral orifice. The woven or braided mesh of the anchor can be formed from elements such as Nitinol and other metals or polymers such as polyester.

In one aspect of the invention, the characteristics of the braid itself may be adjusted to provide differing physical properties in various portions of the structure. Specifically, it would be desirable to have the “rim” of the cup structure that engages in tissue contact with the bladder, be woven with a construction that is more flexible and thus softer than the rest of the structure, the stiffer balance of the structure determining the retention force of the anchor. Thus, both lack of tissue irritation and appropriate retention force may be obtained simultaneously, unlike with a homogeneous structure.

In another aspect of the invention, the same effect may be accomplished by using wires of differing cross-section for different portions of the anchor. For example, the same wire may have a section that is flattened for maximum area in tissue contact and flexibility in one dimension, but retain its circular cross-section where stiffness is required.

In another aspect of the invention, the softer or more flexible braid section is extended further proximal to the tissue contact area. The additional flexibility of this section will add to the capability of the anchor to adapt to positional and other changes in length of the ureter, serving as a shock absorber and further limiting impact at the tissue surface. To the extent that the bladder anchor performs as a shock absorber, this will also limit tension on the kidney coil anchor at the other end of the stent, and possibly provide additional tissue sparing benefits in that location.

In another aspect of the invention, it may be that in certain applications that the ratio of tissue sparing, retention force, and shock-absorption cannot be satisfied by different physical braid configurations alone. In such a situation, a softer tissue-contact region of the braid may be attached to a stiffer retention section by an internal elastic member, thus providing linearly increasing resistance to deformation into the UO in addition to that provided by the braid itself.

In another aspect of this invention, two bladder anchors are arranged sequentially at the end of the stent tubing. The anchor typically in contact with the bladder wall is very soft and functions as a shock absorber and an anchor sufficient for retention of the stent in the bladder under normal circumstances. The second anchor is significantly stiffer and provides higher retention force only once the first anchor is fully collapsed against the bladder wall when unusually stressed such what may occur in an accident or a fall by the patient.

In a still further aspect of the invention, a means to slow or to prevent calcification of the mesh is described. Calcification of ureteral stents in general is a significant problem, particularly when a stent is needed for chronic problems rather than placed for several days after a procedure to ensure ureteral patency. This can result in increased discomfort, obstruction, and difficulty in removal, and has been shown to be a result secondary to formation of a bacterial biofilm on surfaces exposed to urine. Metallic silver has been shown to have broad spectrum antibacterial properties and has seen many applications in medicine from use as a silver foil covering for surgical lesions or bandages to ionic silver sources for transcutaneous external fixation and of course silver nitrate and silver sulfadiazine, both potent broad spectrum, topical antibiotics. Use of the appropriate silver alloy for the mesh wires can give additional protection from biofilm formation, as well as could silver coating or plating on other alloys such as Nitinol. Metallic silver can also be coated over polymeric materials via electroless plating or by sputtering.

### BRIEF DESCRIPTION OF THE DRAWINGS

FIG. 1 is a typical self-expanding mesh stent designed to keep a blood vessel or other body lumen patent and constructed of woven metallic wires without anchoring structures.

FIG. 2 is the stent which has been stretched axially to reduce its diameter for placement.

FIG. 3 is a ureteral stent with an ellipsoidal self-expanding anchor as described in U.S. Pat. No. 6,395,021

FIG. 4 is a ureteral stent with ellipsoidal self-expanding anchor placed in the kidney, ureter, and bladder.

FIG. 5 is an enlarged view of an ellipsoid mesh anchor adjacent to the bladder tissue and UO.

FIG. 6 is an enlarged view of a cup or conical shaped mesh anchor of the present invention.

FIG. 7 is an enlarged view of a truncated cup or conical mesh anchor of the present invention.

FIG. 8 is a stent of the present invention with a pigtail coil anchor for the renal pelvis of the kidney and a cup or conical shaped mesh anchor for the bladder.

FIG. 9 is an enlarged view of the anchor placed in the renal pelvis end of the stent.

FIG. 10 is an enlarged sectional view of a cup or conical shaped mesh anchor at the bladder end of the stent of the present invention.

FIG. 11 is a stent of the present invention in position in the ureter

FIG. 12 is an enlarged sectional view of a truncated cup or conical shaped mesh anchor at the bladder end of the stent of the present invention showing clearance between the anchor and the bladder wall BW and the ureteral orifice UO.

FIG. 13 is an enlarged sectional view of a truncated cup or conical shaped mesh anchor at the bladder end of the stent of the present invention. This also shows the embodiment having an internal elastic band or member connecting the bond between anchor and shaft to the most distal and stiffer portion of the anchor.

FIG. 14 is a stent of the present invention having multiple mesh anchors at the bladder end. The two anchors having differing characteristics to each optimize either comfort or retention force.

### DETAILED DESCRIPTION OF THE PRESENTLY PREFERRED EMBODIMENTS

Some embodiments of the present invention are illustrated as an example and embodiments are not limited by the figures of accompanying drawings:

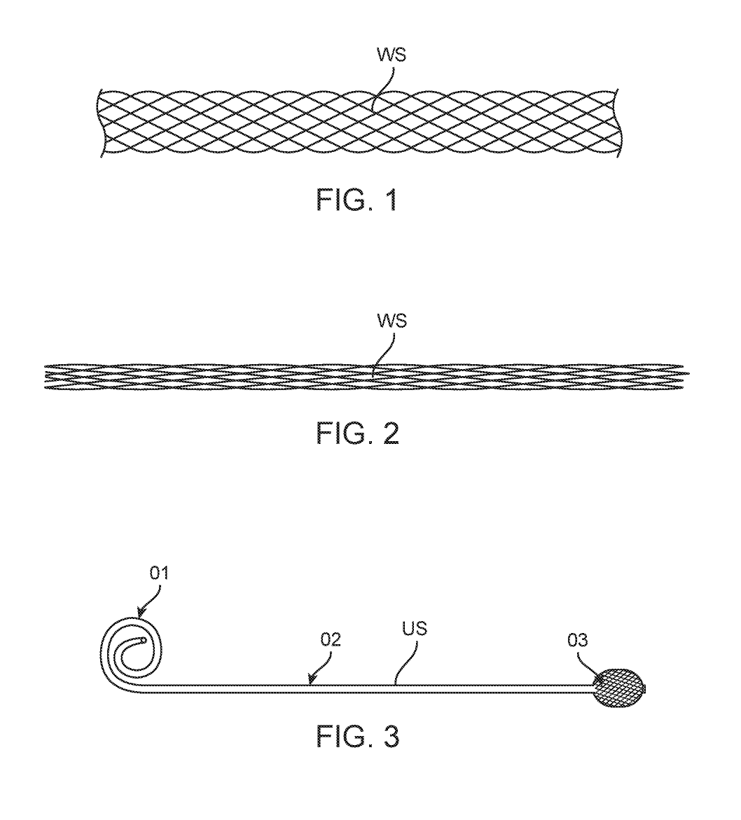

FIG. 1 FIG. 1 illustrates the braid construction of a flexible, self-expanding stent such as the Wallstent® manufactured by Boston Scientific Corporation WS. The braid is made out of a flexible small diameter metal wire such as shape memory Nitinol. The combination makes this particular braid very soft and flexible. In this particular braid construction, the braid is created on a slightly larger mandrel than the desired final outer diameter. The wire size is in the range of outer diameter 0.001″ to 0.007″. The pick count of the braid is selected between 20 PPI to 80 PPI. The braid configuration and wire parameters are selected to make sure the braid will form a desired cylindrical shape having a diameter sufficient to slightly dilate a target lumen and, to attempt to maintain its position in the lumen despite fluid flow, patient movement, and, with most difficulty, peristalsis.

FIG. 2 illustrates the braid's (from FIG. 1) ability to be stretched axially to form a lower profile for placement that is significantly less than its expanded diameter. This allows delivery of the stent through compatible accessories such as a delivery catheter, an endoscope or a sheath (not shown). The braid pick count will be reduced during the stretching of the braid, but a minimum, typically 5 PPI, is selected to ensure self-expansion after the braid is stretched to its lowest desired profile.

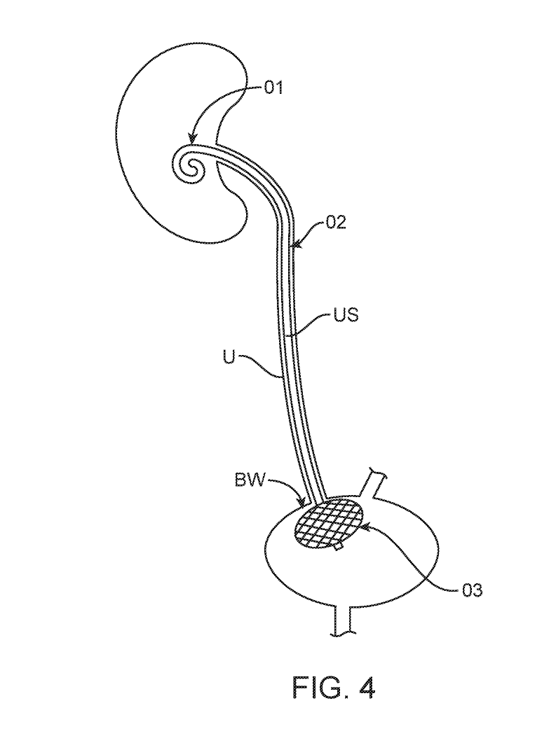

FIG. 3 illustrates a device using the braid in FIG. 1 which has been shape-set to cause it to increase its diameter to a pre-set configuration when no longer stretched axially or constrained by a lumen. Depending on the shape that was set previously, this increase in diameter may form a sphere or ellipsoid as shown in the prior art. A complete assembly of a ureteral stent US is shown with a traditional pigtail kidney anchor 01 at one end, a soft polymer shaft 02 and an ellipsoidal bladder B anchor 03 made out of the same type of braid described above in FIG. 1 and FIG. 2

FIG. 4 illustrates the ureteral stent US of FIG. 3 in place in the ureter U. The proximal end of the stent having the mesh bladder anchor 03 is shown slightly compressed against the bladder wall BW as if there is some positional stretching of the ureter occurring.

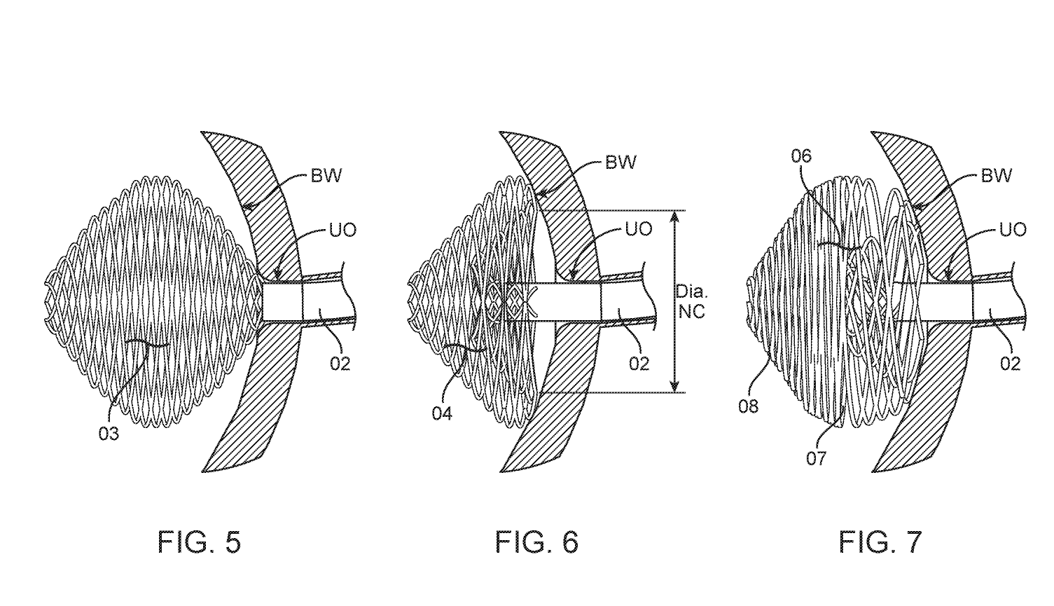

FIG. 5 illustrates an enlarged view of the ellipsoidal mesh anchor 03 of FIG. 3 and FIG. 4, shown in contact with the ureteral orifice UO and the bladder wall BW adjacent to the ureteral orifice. If the mesh in this construction is soft, it can extrude into the UO when under tension by the shaft 02, then pull back when tension is relieved, thus abrading the delicate mucosa inside the ureter. If stiff enough for good retention and to avoid extrusion, the mesh can abrade the sensitive mucosa and enervated tissue of the bladder wall BW directly adjacent to the UO

FIG. 6 illustrates an enlarged view of a cup or conical shaped mesh anchor of the present invention 04, with its concave surface 05 facing the bladder wall BW and UO. As stretching of the ureter puts tension on the shaft and pulls the anchor against the bladder wall BW, the circular bearing surface of the anchor is separated from the ureteral orifice UO and the enervated bladder wall BW adjacent to the UO. Some of the protruding shaft 02 may be pulled into the UO, but not the mesh portion of the anchor 04. The internal diameter of the bearing surface NC, which defines the non-contact area, should be at a minimum 3X the diameter of the stent shaft 02.

FIG. 7 illustrates an enlarged view of an alternative embodiment of a cup or conical shaped mesh anchor 06 which has been shortened or compressed to provide increased bearing surface against the bladder wall BW. This will distribute the force on the bladder wall by the anchor over a wider area and thus reduce trauma to the tissue. Increased surface contact will decrease the non-contact area adjacent to the enervated bladder wall BW so it is important to adjust the diameter of the anchor to maintain a minimum non-contact area of approximately 3 times the diameter of the UO by adjusting the OD of the anchor. Anchor 06 also illustrates the use of varying the weave of the mesh to change the physical properties in different portions of the anchor. A low pick count in the region contacting the bladder wall 07 results in an open, softer, more compressible mesh, which acts as a shock absorber. A relatively high pick count in the proximal area of the anchor 08 results in a denser weave, and a stiffer mesh which provides high compressive force and retention when needed.

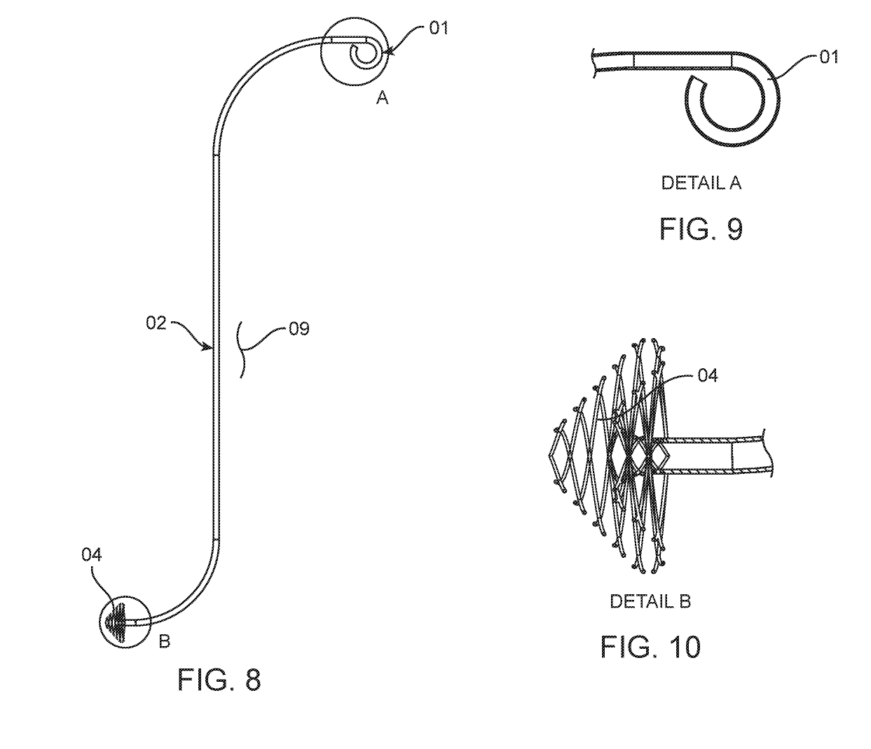

FIG. 8 illustrates a ureteral stent of the present invention 09 having a pigtail anchor 01, a polymer shaft 02, and a cone shaped mesh anchor 04.

FIG. 9 illustrates an enlarged sectional view of the pigtail anchor 01.

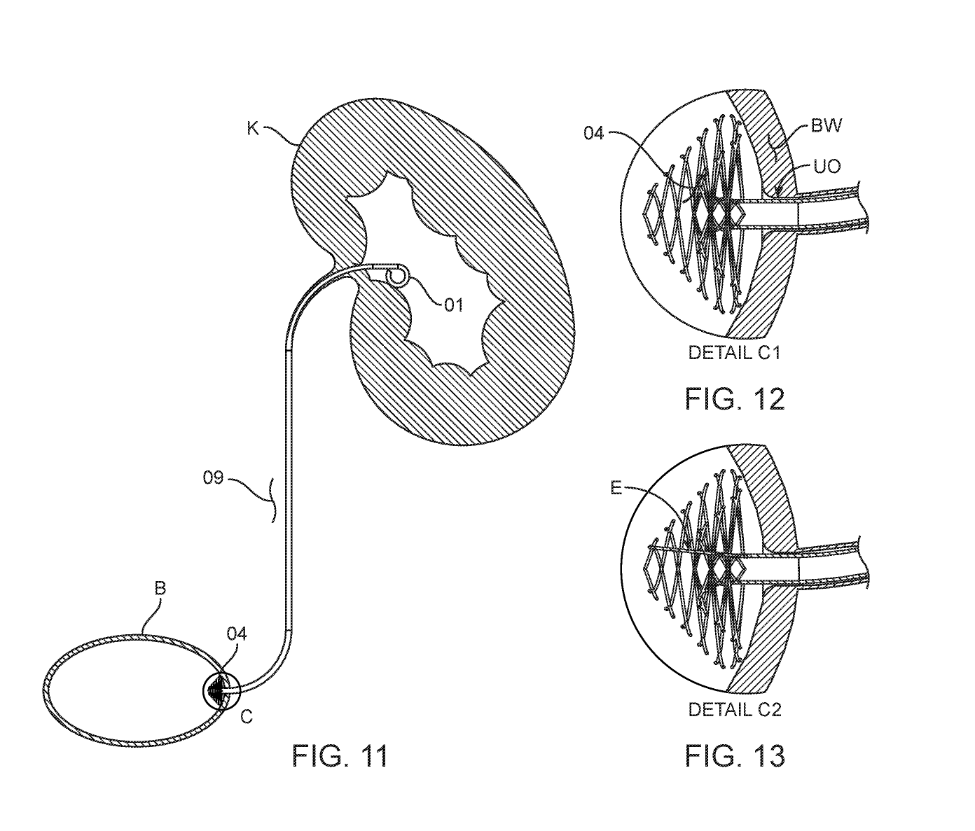

FIG. 10 and FIG. 12 illustrate an enlarged sectional view of the cone shaped mesh anchor 04. FIG. 12 shows that anchor when deployed in the bladder B. Note how the tissue contact surface of the anchor avoids contact with the ureteral orifice UO or the immediate surrounding bladder wall BW when deployed as illustrated.

FIG. 11 illustrates a ureteral stent of the present invention 09 in position in the ureter U, having a deployed pigtail anchor 01 in the renal pelvis of the kidney K and a view of the cone shaped mesh anchor 04 as deployed in the bladder B.

FIG. 13 illustrates an enlarged sectional view of an additional embodiment of the cone shaped mesh anchor 04. This embodiment additionally incorporates an elastic member E internally connecting the distal end of the anchor to the proximal end of the shaft. This pre-loads the compression of the mesh, increasing its retention force, while also helping to maintain the cup or cone shape.

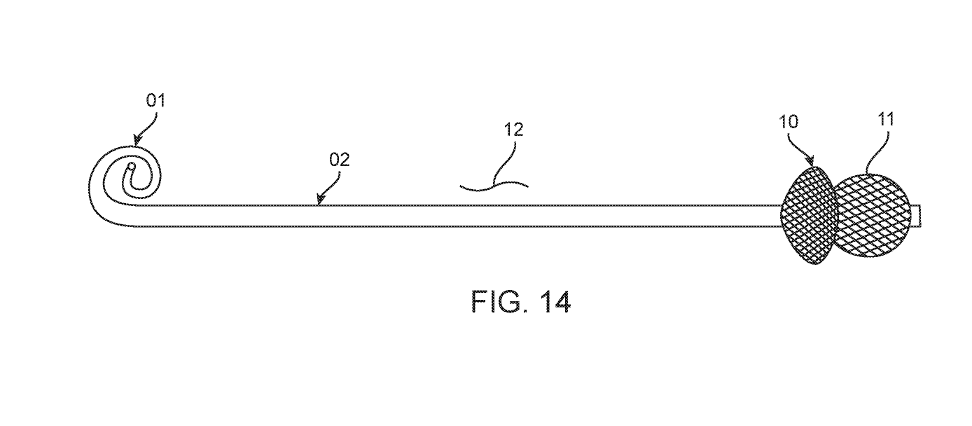

FIG. 14 illustrates an additional embodiment of the present invention, consisting of two individual mesh anchors 10 and 11 mounted congruently at the bladder end of a ureteral stent 12. Similar to the anchor of FIG. 7, the mesh anchor 11 closest to the ureteral orifice UO and typically in contact with bladder wall BW is formed of a softer, better shock absorbing braid, and the mesh anchor 10, which is not typically in contact, is formed of a stiffer braid and provides a higher level of retention force. With two separate anchors, one has additional means such as different wire diameters, wire profiles, and materials to provide different properties, rather than only adjusting the pitch/density of the mesh. Differing wire profiles (not shown) could provide additional advantages such as use of flattened profile wire in the contact anchor 11 to increase surface area in tissue contact and/or to enhance flexibility in one direction, and decrease flexibility in another.

Although a number of exemplary embodiments of the invention have been shown and described, many other changes, modifications, substitutions will now be apparent to those of ordinary skill in the art, without necessarily departing from the spirit and scope of this invention as set forth in the following claims.

What is claimed is:

1. A method for deploying a ureteral stent, said method comprising:
Advancing the distal anchoring device at the distal end of the ureteral stent into a patient's kidney;
Expanding the proximal anchoring device in the bladder wherein a surface of the anchoring device is at risk of engaging the ureteral orifice or immediately adjacent tissue when the anchor is fully deployed;
Pre-shaping or deforming the expanded anchoring device to reduce the risk of the expanded anchoring device engaging the ureteral orifice or immediately adjacent tissue when the stent is fully deployed.

2. A method as in claim 1 wherein the bladder-end anchor is fabricated from a woven mesh that acts as a shock absorber.

3. A method as in claim 1 wherein expanding the proximal anchoring device comprises releasing the proximal anchoring device from constraint so that said proximal anchoring device self-expands in the bladder.

4. A method as in claim 2 wherein the woven mesh material has antibacterial properties or coating to avoid encrustation

5. A method as in claim 4 wherein the antibacterial mesh material is a silver alloy or silver coating.

6. A method as in claim 1 wherein pre-shaping and deforming comprises everting a distal surface of the expanded proximal anchoring device into a cup-shaped form to create a contact region with the bladder wall spaced radially outwardly from the shaft of the ureteral stent to create a non-contact region surrounding the ureteral orifice.

7. A method as in claim 6 wherein the contact region is an annular ring surrounding the ureteral stent shaft, with an inner non-contact diameter at least three times the outer diameter of the stent shaft.

8. A method as in claim 6 wherein the contact region of the cup-shaped form is softer and more flexible than the balance of the anchor.

9. A method as in claim 1 wherein there is a second proximal anchor on the stent having higher retention force and located proximal to a softer anchor in normal contact with the tissue.

10. A method as in claim 1 and claim 6 wherein retention force of the proximal anchor in the bladder may be made variable by use of an stretchable internal resistance member

11. A method as in claim 10 wherein the stretchable internal resistance element increases anchor stiffness and retention force as the stent is pulled harder up the ureter.

12. A ureteral stent having a proximal anchor in the bladder with reduced tissue irritation and improved shock-absorbing characteristics, said ureteral stent comprising:
a stent body or shaft;
a proximal anchor in the bladder formed from a woven mesh at the proximal end of the stent body or shaft, wherein the proximal anchor can be stretched axially to reduce diameter during placement, and radially expand after placement;
wherein the proximal anchor is configured to have an initial self-expanded conformation pre-shaped or everted into a flexible concave cone or cup-shape having an annular contact region shaped to avoid contact pressure against the ureteral orifice or bladder tissue immediately adjacent to the ureteral orifice when the stent is placed therethrough.

13. A bladder-end anchor as in claim 12, wherein the woven mesh has different physical characteristics in the bladder tissue contact area than in the balance of the anchor.

14. The woven mesh of the anchor in claim 13, wherein the mesh in tissue contact is softer than the balance of the anchor.

15. The woven mesh of the anchor in claim 13, wherein the wires of the mesh in tissue contact are flat, while the remaining mesh wires are round.

16. The woven mesh of the anchor in claim 13, wherein the mesh in contact with the tissue has a more open weave than the remaining mesh.

17. The bladder-end anchor as in claim 12, wherein the flexible cone or cup shape in contact with the bladder tissue has an inner diameter of at least three times the diameter of the stent shaft where there is no contact with the ureteral orifice or bladder tissue.

18. A bladder-end anchor as in claim 12, wherein the strands of the mesh are made from Nitinol, stainless steel, or a silver alloy.

19. A bladder-end anchor as in claim 12, wherein the strands of the mesh are made of a polymeric material such as a fluoropolymer, polyethylene or polyester.

20. A bladder-end anchor as in claim 12, wherein there is an internal resistance member that increases anchor stiffness as the anchor is pulled towards the ureteral opening.

21. An internal resistance member for a bladder-end anchor as in claim 20 consisting of an elastic cord or spring between the most proximal end of the anchor and the attachment of the anchor to the body of the stent.

22. A multi-part anchor where a second stiffer anchor is formed proximally to the anchor in claim 12, this anchor not typically in direct tissue contact, and having higher retention force than the anchor in typical tissue contact.
